(19) United States
(12) Patent Application Publication
(10) Pub. No.: US 2018/0028593 A1
(43) Pub. Date: Feb. 1, 2018

(54) TERMINAL NANOFILTRATION OF SOLUBILIZED PROTEIN COMPOSITIONS FOR REMOVAL OF IMMUNOGENIC AGGREGATES

(71) Applicant: BOREAL INVEST, Boulogne Billancourt (FR)

(72) Inventor: Michel MORRE, Boulogne-billancourt (FR)

(21) Appl. No.: 15/127,184

(22) PCT Filed: Mar. 19, 2015
(86) PCT No.: PCT/IB2015/052023

§ 371 Date: Sep. 19, 2016

Related U.S. Application Data
(60) Provisional application No. 61/968,850, filed on Mar. 21, 2014.

(51) Int. Cl. A61K 38/01; C07K 1/34

(52) U.S. Cl. CPC A61K 38/012; C07K 1/34

(57) ABSTRACT

The present invention relates to a method for preparing a pharmaceutical composition comprising a protein active ingredient and having a reduced amount of protein aggregates, the said method comprising performing a step of nanofiltration of a starting composition comprising the said protein active ingredient in a solubilized form, whereby the said pharmaceutical composition is obtained.

### FIELD OF THE INVENTION

This invention relates to nanofiltered protein therapeutic compositions having a reduced content in protein aggregates (auto and hetero-aggregates) and consequently a decreased immunogenic potential. The invention also discloses methods for the removal of these aggregates at the end of the manufacturing process and for the production of said protein compositions. This proceeds through solubilization and nanofiltration of the pharmaceutical protein composition in a specific formulation buffer, optionally followed by freeze drying. More particularly, the invention relates to therapeutic proteins with a high potential to trigger anti-protein immunogenicity. This includes proteins highly susceptible to form non-covalent aggregates, proteins used for chronic therapies, proteins with an immuno-activating activity.

### BACKGROUND OF THE INVENTION

Many therapeutic proteins are immunogenic in patients and this immunogenicity is related to the residual presence of protein aggregates, mostly non covalent, in the pharmaceutical protein preparation

More and more therapeutic proteins, often recombinant and monoclonal antibodies are developed and introduced to market for their therapeutic applications. These agents are prone to aggregation, mostly through various hydrophobic molecular interactions. Among therapeutic recombinant proteins the more hydrophobic are the more prone to aggregation. This is for instance the case of the blood coagulation factors like anti-hemophilic Factor VIII and Factor VII. Various methods have been used in the past aimed at minimizing these hydrophobic interactions and the resulting presence of non-covalent molecular aggregates.

With the development of immunotherapy, many immunoactive agents are developed. Cytokines and interferons are used for their specific abilities to favor the growth, differentiation and/or activation of specific cells of the immune system. New Monoclonal antibodies, also called “check point blockers”, are developed for their ability to counteract the anergy of immune system cells observed in tumors. Because these immunotherapeutic agents tend to trigger, increase and or prolong the immune responses, they also tend to favor the production of an immune response against themselves. It is now well established that non-covalent molecular aggregates of these therapeutic proteins are immunogenic, but in addition the careful clinical detection and measure of the anti-protein antibodies clearly demonstrates that this immuno-active agents are more prone to trigger anti-protein immunogenicity than many other therapeutic proteins which do not interfere with the immune system. The immuno-stimulating activity of these agents easily explains their increased risk of immunogenicity and justifies to develop and implement new methods to reduce these non-covalent aggregates to very low levels and stabilize the product to block any risk of regeneration of these aggregates.

It is now well established that patients treated with recombinant proteins or monoclonal antibodies used for therapeutic purposes are at risk of developing anti-protein immune reactions among which the development of anti-protein antibodies. These antibodies can bind the therapeutic protein. This could modify the protein pharmacokinetic and secondarily its profile of activity. These antibodies can also directly neutralize the activity of the protein and block its therapeutic activity. Finally these antibodies are also susceptible to neutralize the activity of the endogenous natural homolog of the protein, leading to critical and sometime fatal toxicity.

The generation of these anti-protein antibodies is not restricted to proteins heterologous to the host. These antibodies have been observed for recombinant proteins with a natural human amino-acid sequence, such as □ and □ interferons, human growth hormone, erythropoietin, platelet growth factor, coagulation factors and various humanized monoclonal antibodies.

Progressively the analysis of multiple sets of clinical data highlighted the high immunogenic potential of large covalent and non-covalent aggregates of therapeutic proteins. Although covalent aggregates of therapeutic proteins have long been suspected of immunogenicity, the use of size exclusion chromatography easily allowed their detection and their elimination. Size exclusion is also able to detect the so-called insoluble aggregates, those that can be removed by simple filtration. But these size exclusion chromatography steps are not able to detect nor to eliminate the non-covalent aggregates. Such aggregates mainly linked by various hydrophobic and/or electrostatic interactions are soluble and quickly disassemble during, but reassemble after size exclusion chromatography (performed either for analytical of for process purposes), which makes them impossible to detect and quantify by size exclusion chromatography. (Barnard et al., 2011, J. Pharm. Sci. 100, 492-503).

Among protein aggregates, non-covalent aggregates of proteins are made of many multimers of the said protein. Occasionally they can associate to micro particles of foreign agents such as glass or rubber particles or more frequently to drops of silicone oil used for syringe lubrification. As used herein, a “non-covalent protein aggregate” is defined as being composed of a multiplicity of protein molecules wherein non-native non-covalent interactions hold the protein molecules together and or to foreign particles, mostly through hydrophobic interactions. Typically, but not always, an aggregate contains sufficient molecules so that it is insoluble; such aggregates are insoluble aggregates. Both soluble and insoluble protein aggregates have an immunogenic potential.

This remarkable immunogenicity of large non covalent aggregates of therapeutic proteins has been extensively reviewed (Rosenberg, 2006); (Carpenter et al., 2010) and more recently during a one day workshop held in 2012 “Predictive science of the immunogenicity aspects of particles in biopharmaceutical products” (Marszal and Fowler, 2012, J. Pharm. Sci. 101, 3555-3559; Bee et al., 2012, J. Pharm. Sci. 101, 3580-3585; Wang et al., 2012, Int. J. Pharm. 431, 1-11; Rosenberg et al., 2012, J. Pharm. Sci. 101, 3560-3567).

As stated above, the classic analytical techniques used to detect the presence of protein multimers (size exclusion chromatography and electrophoresis) are inefficient at detecting these non-covalent aggregates because these techniques either exclude or resolve the non-covalent aggregates during the analytical process.

A set of new analytical techniques progressively appeared to detect these aggregates (Zölls et al., 2012, J. Phalli′. Sci. 101, 914-935; den Engelsman et al., 2011, Pharm. Res. 28, 920-933). In our hands, the most convenient and reliable technique was the micro-flow imaging (MFI), which allowed the simultaneous counting and sizing of these aggregates most commonly observed in the sub visible micrometer range (0.2 to 10 μm; occasionally up to 50 μm) (Sharma et al., 2010a, AAPS J. 12, 455-464; Sharma et al., 2010b, J. Pharm. Sci. 99, 2628-2642). Nevertheless, testing recombinant proteins with appropriate methods for the detection of sub visible particles, i.e. in the range of 0.1 μm to 50 μm, reveals the presence of various amounts of these immunogenic covalent and non-covalent aggregates.

A method for the removal of soluble aggregates from protein therapeutics and prevent their regeneration with time would thus contribute significantly to the safety of therapeutic proteins.

Various process operations for decreasing the therapeutic protein contamination by non-covalent protein aggregates have been described in the art. However, these prior art methods did not prove to reduce the presence of protein aggregates to a point sufficient to abolish the generation of anti-protein immunogenicity upon administration to the patients.

The correct and full natural refolding of a recombinant protein tend to expose the hydrophobic surfaces of the protein to the inner core of the folded protein, which in turn exposes the hydrophilic surface to the outside. This by itself minimizes the possibility for hydrophobic interactions with other molecules of the same protein thereby minimizing the formation of aggregates. This led the biotech industry to check the correct disulfide bridging and correct conformation of recombinant proteins. This technology was used to minimize the immunogenicity of recombinant IL-7 immunogenicity (U.S. Pat. No. 7,585,947).

The optimization of the protein glycosylation on its specific glycosylation sites, generates an hydrophilic protection which also minimizes the chances of inter molecular hydrophobic interactions. Within the glycosylation profile, increasing the degree of sialylation of the glycosylated proteins contributes to increase its electric charge and further decreases the probability for hydrophobic interaction (U.S. Pat. No. 8,034,327).

All these measures taken to minimize hydrophobic interactions are efficient to decrease the presence of non-covalent protein aggregates. But for protein highly susceptible to anti-protein immunogenicity, these preventive measures do not suffice to totally eliminate protein aggregates and the subsequent generation of anti-protein antibodies.

Previous works have determined that application of high pressure to protein composition is able to resolve the high molecular weight aggregates (US patent applications no US 2008/0161242, no US 2012/0070406; no US 2013/0058895). At laboratory scale, the use of a high pressure treatment step appears efficient for removing protein aggregates. However, such a step of high pressure treatment requires an additional and unusual operation to an industrial process flow of operations. Together this does not fully guarantee the reassembling of non-covalent aggregates with time.

Altogether the current knowledge teach us that most pharmaceutical protein compositions contain significant amount of non-covalent aggregates of said proteins, occasionally combined to other particular contaminants. These aggregates are potentially immunogenic and have still a higher risk of immunogenicity when said proteins belong to the broad family of immuno-active agents like cytokines (interleukins or interferons) or activating monoclonal antibodies.

Previous methods used for the removal of these soluble non-covalent aggregates did not lead to a sufficient removal of these immunogenic contaminants or did not demonstrate how to prevent their regeneration with time.

There is a need in the art for further methods aimed at removing soluble non-covalent aggregates in processes of manufacturing pharmaceutical compositions, which are alternative methods or improved methods as compared to the methods known in the art.

### SUMMARY OF THE INVENTION

The present invention discloses how a new combination of operations easy to conduct at industrial scale can contribute to the removal of these non-covalent aggregates and prevent their regeneration with time.

The standard final steps of production of therapeutic proteins consist in ultra-filtration/dia-filtration. This dia-filtration is used to place said proteins in their final formulation at the desired concentration. This operation terminates the production of the “drug substance”. Said drug substance could then be stored liquid or frozen for days or weeks. Later on some further additives could optionally be mixed with the drug substance to produce the drug product ready for filling operations. Not only these last operations do not contribute to remove aggregates but they are also known for their potential to generate molecular aggregates which will contaminate the “drug product” and are known to further facilitate the generation of new aggregates through a process known as nucleation.

The present invention relates to a method for preparing a pharmaceutical composition comprising a protein active ingredient and having a reduced amount of protein aggregates, the said method comprising performing a step of nanofiltration of a composition comprising the said protein active ingredient in a solubilized form, whereby the said pharmaceutical composition is obtained.

The composition to be nanofiltered may also be termed “intermediate composition” or “starting composition” herein, especially in embodiments of the invention's method wherein the composition to be nanofiltered consists of a composition resulting from a multi-step process of preparing a protein-containing composition, e.g; a multi-step process of obtaining a composition comprising a purified protein of interest. Thus, the terms “intermediate composition” and “starting composition” may be used interchangeably herein”.

In some embodiments of the method above, the nanofiltration step is performed by using a nanofiltration device, which encompasses a nanofiltration membrane, having a mean pore size of less than 100 nm, advantageously a mean pore size of less than 30 nm, and preferably a mean pore size ranging from 10 nm to 30 nm.

In some embodiments of the method above, the said pharmaceutical composition is introduced in a pharmaceutical container after the nanofiltration step. Storage at this stage should be avoided or minimized in which case frozen storage is preferable.

In some embodiments of the method above, the pharmaceutical composition is subjected to a step of freeze-drying after the nanofiltration step.

In some embodiments of the method above, the nanofiltration step is performed by using a nanofiltration material (membrane, hollow fiber, resin) having a mean pore size of less than 100 nm, advantageously a mean pore size of less than 30 nm, and preferably a mean pore size ranging from 10 nm to 30 nm.

In some embodiments of the method above, the said pharmaceutical composition is introduced in a container after the nanofiltration step, which encompasses introduction of the pharmaceutical composition directly subsequently to the nanofiltration step.

In some embodiments of the method above, the pharmaceutical composition is subjected to a step of freeze-drying after the nanofiltration step, which encompasses performing a freeze-drying step of the pharmaceutical composition directly subsequently to the nanofiltration step.

In some embodiments of the method above, the said intermediate composition has a pH value selected in a group comprising (i) a pH value of 0.2 pH units or more higher than the isoelectric point of the said protein active ingredient, and (ii) a pH value of 0.2 pH units or less lower than the isoelectric point of the said protein active ingredient.

In some embodiments of the method above, the said composition to be nanofiltered has a pH value of 0.2 pH units or less lower than the isoelectric point of the said protein active ingredient.

In some embodiments of the method above, the said composition to be nanofiltered comprises at least two amino acids having opposite charges, advantageously one or more basic amino acid and one or more acidic amino acid, and preferably arginine and glutamate.

In some embodiments of the method above, the one or more basic amino acid is selected in a group comprising L, D, or LD arginine, lysine, histidine and a charged analog thereof such as homoarginine, canavanine, ornithine, oxalysine, or other oxo or thio analogs

In some embodiments of the method above, the one or more acidic amino acid is selected in a group comprising L, D, or LD aspartate, glutamate and a charged analog thereof such as pyroglutamate or other oxo or thio analogs. In some embodiments of the method above, the said at least two amino acids having opposite charges comprise arginine and glutamate.

In some embodiments of the method above, each charged amino acid is present in the intermediate composition at a concentration ranging from 20 mM to 200 mM, and preferably at a concentration ranging from 50 mM to 100 mM.

In some embodiments of the method above, the molar ratio of the acidic amino acid to the basic amino acid ranges from 0.3 to 3.

This invention also pertains to a method for preparing a therapeutic protein composition, and especially a therapeutic composition as described above, comprising the following steps:

a) solubilizing the purified therapeutic protein in a pharmaceutically acceptable carrier,

b) treating the solubilized protein composition by nanofiltration prior to filling in its therapeutic container without adding any other component to the formulation,

c) optionally freeze drying the nanofiltered protein composition dispensed into the therapeutic container, in which case the freeze dried product will have to be reconstituted with a pharmaceutically acceptable diluent prior to its administration.

In some embodiments of the method for preparing a therapeutic protein composition described above, the step of nanofiltration is performed with an industrial scale device similar to the devices commonly used for viral clearance in biotechnical productions.

In some embodiments of the method for preparing a therapeutic protein composition described above, the nanofiltration is applied to the protein composition to be nanofiltered shortly after its solubilization in its final formulation.

In some embodiments of the method for preparing a therapeutic protein composition described above, the amount of aggregated protein in the nanofiltrate is measured by a method selected from the group consisting of analytical ultracentrifugation, size exclusion chromatography, field flow fractionation, light scattering, light obscuration, nano-particles tracking analysis and/or preferably micro flow imaging.

In some embodiments of the method for preparing a therapeutic protein composition described above, the protocol of freeze drying is optimized to minimize the regeneration of micrometric aggregates.

This invention also relates to a pharmaceutical composition that is obtained according to the method described above.

This invention also concerns a pharmaceutical composition having a reduced content in subvisible micrometric protein aggregates of a size ranging from 0.1 μm to 50 μm.

This invention also pertains to a solubilized therapeutic protein composition treated by nanofiltration to reduce its content in subvisible micrometric (0.1 μm to 50 μm) protein aggregates.

In some embodiments, the nanofiltered protein composition is dispensed into pharmaceutical containers and optionally freeze dried for storage.

In some embodiments of the therapeutic protein composition described above, the concentration of large micrometric protein aggregates (3 to 30 μm in size) detected by microflow imaging is reduced by at least 75% in comparison to the same composition not treated by nanofiltration.

In some embodiments of the therapeutic protein composition described above, the concentration of large micrometric protein aggregates (3 to 30 μm in size) detected by microflow imaging remains reduced by 70% when stored at 4° C. for one month.

In some embodiments of the therapeutic protein composition described above, the pharmaceutically acceptable carrier includes at least two oppositely charged amino acids, at least one acidic and one basic, preferably arginine and glutamate.

In some embodiments of the therapeutic protein composition described above, the basic amino-acid is chosen among arginine, lysine, histidine or their various charged synthetic analogs and the acidic amino-acid is chosen among aspartate, glutamate or their various charged synthetic analogs.

In some embodiments of the therapeutic protein composition described above, the pharmaceutically acceptable carrier also contains neutral amino-acids like glycine, alanine, leucine or isoleucine, and/or hydroxyl amino-acids like serine or threonine, the total molarity of which will remain below the molarity of the charged amino acids.

In some embodiments of the therapeutic protein composition described above, the pharmaceutically acceptable carrier also contains a tensioactive agent like Polysorbate 20 or 80.

In some embodiments of the therapeutic protein composition described above, the protein is endogenous to the species of the individual.

In some embodiments of the therapeutic protein composition described above, the protein is a cytokine: an interleukin, such as IL-7, IL-2, IL-21, IL-15, IL-12, or an interferon, such as interferon alpha, beta or gamma and their close analogs.

In some embodiments of the therapeutic protein composition described above, the protein is a fusion protein comprising a cytokine or the soluble receptor of a cytokine (interleukin or interferon) and the Fc fragment of an immunoglobulin.

In some embodiments of the therapeutic protein composition described above, the protein is an immuno-activating monoclonal antibody like anti-PD1, anti-PDL1, anti-CTLA-4, anti-Lag3, anti-Tim3, anti-TGFβ.

In some embodiments of the therapeutic protein composition described above, the recombinant protein is a hormone, a growth factor or an enzyme used for chronic therapy.

In some embodiments of the therapeutic protein composition described above, the recombinant protein is a human growth hormone or an anti-hemophilic factor like factor VII or VIII.

### BRIEF DESCRIPTION OF THE FIGURES

FIG. 1: Modification of the Terminal Steps of the production process of a therapeutic protein.

In the new process design a specific step of nanofiltration is added after the ultrafiltration/diafiltration (UF/DF). The UF/DF is used to place the protein in its final composition containing the opposite charged amino-acids, the nanofiltration is used to eliminate the aggregates. Contrary to the old process in the new process there is no addition of any new components in the formulation after the UF/FD.

FIG. 2:

FIG. 2 a Example of an experimental monoclonal antibody “A” stirred for 3 days or submitted to 4 freeze thaw cycles compared to the same antibody nano-filtered, and the same antibody nanofiltered and stored 3 months at 4° C. showing the stability of the composition and non-regeneration of aggregates.

FIG. 2 b Example of an experimental monoclonal antibody “B” heated at 60° C. for 60 min, compared to the same antibody un stressed before or after nano-filtration. The same antibody nanofiltered and stored 3 months at 4° C.

Both antibodies have been diafiltered before to be placed in the buffers described in the examples, tested at a dilution of 1 mg/mL, the number of subvisible particles/mL is expressed Log 10.

FIG. 3:

Aggregate reduction through nanofiltration of a solubilized composition of glycosylated IL-7

3a effect of a final nanofiltration compared to a nanofiltration operated before the UF/DF

3b effect of a final nanofiltration on the liquid drug substance stored at 4° C. for 1 month

The IL-7 drug product was tested at 2 mg/mL in the buffer composition described in the example.

FIG. 4: Preparation of an Interferon beta composition: a commercial preparation devoid of serum albumin was diafiltered to be placed in the buffer composition described in the example. The composition was heated at 60° C. for 60 min and results were compared to the same Interferon beta composition before and after Planova filtration or after storage at 4° C. The IFN drug product was also tested at 0.2 mg/mL

### DETAILED DESCRIPTION OF THE INVENTION

All publications and patents mentioned herein are hereby incorporated by reference in their respective entireties. The publications and patents disclosed herein are provided solely for their disclosure. Nothing herein is to be construed as an admission that the inventors are not entitled to antedate any publication and/or patent, including any publication and/or patent cited herein.

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

This invention provides for a method for removing protein aggregates from a composition aimed at being administered to an individual in need thereof. More precisely, the present invention provides a method for removing therapeutic protein aggregates at a final step of preparing a pharmaceutical composition.

The preparation of pharmaceutical compositions comprising one or more protein(s) as the active ingredient(s) typically comprises a plurality of steps, including mainly purification step(s), viral inactivation or viral removal step(s) and formulation step(s).

Typical protein purification processes aimed at preparing purified protein-containing compositions, which includes pharmaceutical compositions, involve multiple chromatography steps in order to meet purity, yield and throughput requirements. The process steps typically involve capture, intermediate purification or polishing, and final polishing. Traditionally, the capture step is followed by one or more intermediate purification or polishing chromatography steps to ensure adequate purity and viral clearance. The intermediate purification or polishing step is typically accomplished via affinity chromatography, ion exchange chromatography, or hydrophobic interaction, among other methods. In traditional processes, the final polishing step may be accomplished via ion exchange chromatography, hydrophobic interaction chromatography, or gel filtration chromatography. These steps are aimed at removing process-related and product-related impurities, including host cell proteins, DNA, leached protein A when present, aggregates, fragments, viruses, and other small molecule impurities from the product stream and cell culture. Typically, such purification processes comprise one or more steps of virus inactivation or virus removing, such as a nanofiltration step for the removal of viruses. Typically, such processes comprise the steps of (i) collecting and optionally clarifying a protein-containing sample, (ii) a capture step, (iii) a viral inactivation step, (iv) one or more intermediate/final polishing steps, (v) a viral removing step which is generally a nanofiltration step, and (vi) an ultrafiltration/diafiltration step. The ultrafiltration/diafiltration step, when present, is aimed at achieving the protein active ingredient concentration and buffer condition before conditioning the final pharmaceutical composition for storage or for its administration to an individual in need thereof.

It is now well established that micrometric protein aggregates within the size range of from 0.1 μm to 50 μm are present in many pharmaceutical compositions comprising therapeutic proteins, which protein aggregates significantly contribute to an undesired immunogenicity of these proteins. The immunogenicity of these therapeutic protein aggregates is mainly illustrated by the production of anti-protein antibodies in individuals to which these pharmaceutical compositions are administered. These anti-protein antibodies may bind to the target therapeutic protein and may neutralize, at least partly, the expected biological activity of the therapeutic protein. It shall be underlined that, when formulated in pharmaceutical compositions comprising therapeutic protein aggregates, even proteins of the self, such as human interleukins, may become immunogenic when administered to a human individual and thus induce the production of anti-protein antibodies.

Although the presence of these therapeutic protein aggregates may not be the only cause of protein immunogenicity, it has been shown by many authors that it is often the main culprit. In these protein aggregates, protein molecules may be associated through covalent bonds or through non-covalent bonds (e.g. hydrogen bonds, Van der Waals forces, etc) involve only the protein molecules or may also include foreign particles like metal or rubber particles or drops of silicone oil.

Former techniques for detecting aggregates, such as standard “light obscuration” and “size exchange chromatography” do not allow to reliably detect and quantify these therapeutic protein aggregates that may be contained in pharmaceutical compositions. However, recent methods now allow a sensitive and reproducible detection of micrometric aggregates, among which methods it may be cited the MicroFlow imaging (MFI) technology, which appears the more convenient to control pharmaceutical compositions. The availability of these recent methods for detecting micro-aggregates, including detecting therapeutic protein micro-aggregates in a composition, now allows for exploring appropriate conditions for preparing pharmaceutical compositions comprising one or more protein(s) as the active ingredient(s) which shall possess a low content in protein aggregates.

The present invention provides for a method aimed at lowering the content of protein-based pharmaceutical compositions in protein aggregates, especially in protein aggregates having a particle size equal to, or higher than, 0.1 μm, which includes protein aggregates having a particle size ranging from 0.1 μm to 50 μm.

Thus, the present invention provides for a novel convenient way of removing most of the protein aggregates that may be contained in a composition aimed at preparing a pharmaceutical composition, through the use of technologies that are familiar for engineers skilled in the pharmaceutical and biotechnological industries.

In some embodiments, the invention's method comprises a step of solubilizing a therapeutic protein in its final buffered formulation, followed by a step of nanofiltration of the said formulation so as to obtain a pharmaceutical composition that may readily be administered to an individual in need thereof, or alternatively that may be stored in appropriate storage conditions before being administered to an individual in need thereof.

Surprisingly, it is shown herein that protein aggregates that are present in compositions for pharmaceutical use may be successfully removed by performing a final step of nanofiltration before conditioning the said compositions for their storage or for their administration to an individual in need thereof.

Then, it is shown herein that, in a method of preparing a pharmaceutical composition comprising a protein as an active agent, subjecting an intermediate composition obtained at the end of the purification/preparation method to a final nanofiltration step before conditioning the resulting pharmaceutical composition allows removing most of the protein aggregates that are present in the intermediate composition.

According to the inventors knowledge, the removing of the protein aggregates by performing a nanofiltration step is expected to result in a pharmaceutical composition having a lower immunogenicity for the administered patient, as compared to the same composition that has not been subjected to this final step of nanofiltration.

It is shown in the examples herein that subjecting a therapeutic composition comprising a protein active ingredient to a final nanofiltration step allows removing a large portion of protein aggregates that are initially present in the said therapeutic composition. The examples herein show that performing such a final nanofiltration step permits reducing the amount of protein aggregates initially present, irrespective of the kind of protein which is used as the active ingredient, i.e. irrespective of the size, molecular weight, charge or other physico-chemical properties of the protein active ingredient. This is illustrated in the examples with therapeutic proteins such as various antibodies and various cytokines.

Particularly, it is shown in the examples herein that protein aggregates are present in compositions comprising proteins of therapeutic interest, which compositions have undergone the conventional steps of capture, viral inactivation, polishing, viral removing (nanofiltration) and ultrafiltration/diafiltration. It is also shown in the examples that subjecting such conventionally prepared compositions to a final step of nanofiltration before conditioning for storage or use allows substantially reducing the amount of protein aggregates. This has been shown herein notably for IL-7-containing compositions and beta interferon-containing compositions.

It is also shown herein that the final step of nanofiltration allows removing or reducing protein aggregates and avoids reformation or regeneration of protein aggregates even after a long period of time of storage of the resulting pharmaceutical composition.

Conversely, it is observed herein that an intermediate nanofiltration step aimed at removing viruses that is found in conventional processes of preparing pharmaceutical compositions does not avoid the presence of significant amounts of protein aggregates in the final formulation.

Without wishing to be bound by any particular theory, the inventors believe that the intermediate nanofiltration step may itself remove at least a portion of the protein aggregates that are present. However, as it has been previously specified herein, the anti-viral nanofiltration step is followed by a plurality of subsequent process steps, which include polishing step(s) and ultrafiltration/diafiltration step(s), in which subsequent steps reformation or regeneration of protein aggregates occurs.

In contrast, the method according to the invention comprises a final step of nanofiltration which is not followed by any subsequent process step, e.g. polishing, ultrafiltration/diafiltration, buffering, etc, before conditioning the resulting pharmaceutical composition for storage or for use. According to the inventors knowledge, this explains why, by performing the invention's method, protein aggregates are definitely removed or reduced in the resulting pharmaceutical composition.

This invention relates to a method for preparing a pharmaceutical composition comprising a protein active ingredient and having a reduced amount of protein aggregates, the said method comprising performing a step of nanofiltration of an intermediate composition comprising the said protein active ingredient in a solubilized form, whereby the said pharmaceutical composition is obtained.

In some embodiments of the method, the nanofiltration step is performed by using a nanofiltration devices, which encompasses a nanofiltration membrane, having a mean pore size of less than 100 nm, advantageously a mean pore size of less than 30 nm, and preferably a mean pore size ranging from 10 nm to 30 nm.

The inventors believe that using a nanofiltration device having a mean pore size of less than 10 nm, although it may be efficient for removing or reducing protein aggregates; may cause process drawbacks such as a clogging of the said device filter which would prevent performing the nanofiltration step in optimal conditions.

It is also believed that using a nanofiltration device having a mean pore size of more than 100 nm would be less efficient since it is not expected that a significant portion of the protein aggregates present in the composition to be processed possess a particle size lower than 100 nm.

Further, according to the inventors knowledge, protein aggregates having a size in the 100 nm range or lower are not quantitatively preponderant and further are not the most immunogenic protein aggregates.

As already specified herein, the nanofiltration step is the final step of a method of preparing a protein-containing pharmaceutical composition, and particularly the final step of preparing a pharmaceutical composition comprising one or more protein(s) as the active ingredient(s).

Thus, according to some embodiments of the method, the said pharmaceutical composition is introduced in a container after the nanofiltration step. The container may be any container for pharmaceutical compositions that are known in the art, which includes polystyrene, polypropylene or glass containers. In some embodiments, the container comprises an amount of pharmaceutical composition corresponding to one dosage unit. In other embodiments, the container comprises an amount of pharmaceutical composition corresponding to a plurality of dosage units.

In some embodiments of the method, the pharmaceutical composition is subjected to a step of freeze-drying after the nanofiltration step.

As it is disclosed throughout the present description, the final nanofiltration step may be performed in optimal conditions wherein the protein of interest comprised in the intermediate composition to be nanofiltered is solubilized in an appropriate buffer solution.

Thus, in some preferred embodiments, the nanofiltration step is performed with an intermediate composition comprising, or consisting of, a specific buffer which favors an optimal solubilization of the protein of interest. Illustratively, it is disclosed herein the preferred use of low ionic strength buffers containing a tandem of opposite charged amino acids like arginine and glutamate, with a pH slightly distant from the isoelectric point of the protein to preserve its electric charge. Such buffers shall preserve the net electric charge of the protein, its colloidal stability thereby minimizing the hydrophobic interactions between the protein molecules and other particles. Not only these buffers favor the resolution of aggregates through nanofiltration but they also prevent the potential regeneration of these aggregates afterwards. This is illustrated by the non significant regeneration of aggregates observed after storage at 4° C.

Consequently, in some embodiments of the method, the said intermediate composition to be nanofiltered has a pH value selected in a group comprising (i) a pH value of 0.2 pH units or more higher than the isoelectric point of the said protein active ingredient, and (ii) a pH value of 0.2 pH units or less lower than the isoelectric point of the said protein active ingredient.

In preferred embodiments of the method, the said intermediate composition has a pH value of 0.2 pH units or less lower than the isoelectric point of the said protein active ingredient.

In other preferred embodiments of the method, the said intermediate composition comprises at least two amino acids having opposite charges, advantageously one or more basic amino acid and one or more acidic amino acid, and preferably arginine and glutamate.

In further embodiments of the method, the one or more basic amino acid is selected in a group comprising L, D, or LD arginine, lysine, histidine and a charged analog thereof such as homoarginine, canavanine, ornithine, oxalysine, or other charged oxo or thio analogs.

In still further embodiments of the method, the one or more acidic amino acid is selected in a group comprising L, D, or LD aspartate, glutamate and a charged analog thereof such as pyroglutamate or other charged oxo or thio analogs.

In some preferred embodiments of the method, the said at least two amino acids having opposite charges comprise arginine and glutamate.

In some preferred embodiments of the method, each charged amino acid is present in the intermediate composition at a concentration ranging from 20 mM to 200 mM, and preferably at a concentration ranging from 50 mM to 100 mM.

In further preferred embodiments of the method, the molar ratio of the acidic amino acid to the basic amino acid ranges from 0.3 to 3.

According to the present invention, the nanofiltration step most preferably consists of the ultimate step of a production process of the protein-containing composition.

In preferred embodiments, the nanofiltration step of the method is performed almost immediately before filling the pharmaceutical containers.

In contrast, standard antiviral nanofiltration steps are performed during the course of the protein purification process and at the latest before the step of ultrafiltration/diafiltration used for final buffer exchange. Here it is essential to protein aggregates removal or reduction that the nanofiltration step be performed at the end of a pharmaceutical composition preparation method, i.e. on the very final step of formulating the protein-containing composition. It is thus essential to the invention's method that further process operations occurring after this final nanofiltration step are absent or minimized, in view of avoiding reformation of protein aggregates. In that view contrary to standard process design, the resulting final nanofiltrate will, in most cases, only be subjected to a dilution step so as to adjust the final protein concentration to the desired concentration for use. In fact this final protein concentration adjustment will also allow the wash of the nanofilter to ensure full recovery of the product at the end of the nanofiltration step. Preferably, long term storage, unless at −20° C., will be avoided or minimized and addition of further compounds to the resulting composition will be avoided.

In some embodiments, the resulting pharmaceutical composition, once introduced in the appropriate containers (vials, syringes), is freeze dried, in which case the freeze drying cycle is performed according to operating conditions suitable for minimizing the generation of aggregates detected by MFI.

All these operations are easy to handle with commonly existing technologies used in the pharmaceutical industry and well known form the one skilled in the art.

The method described herein is advantageously used for protein compositions highly susceptible to anti-protein immunogenicity like hydrophobic proteins susceptible to generate aggregates, which encompass cytokines and monoclonal antibodies with immuno-stimulating activities, as well as proteins used for chronic pharmacological treatments such as hormonal or enzyme replacement therapies.

The method of the present invention can be used to prepare compositions comprising therapeutic recombinant proteins having a reduced content in protein aggregates and thereby possess low immunogenic properties. The invention's method is especially useful for the removal of immunogenic soluble protein aggregates, which include covalent and non-covalent protein aggregates, within the context of an industrial process of production of protein-containing compositions, especially pharmaceutical compositions, while avoiding their spontaneous regeneration with time.

More specifically, the method described herein discloses a new way of finishing the production process of compositions comprising therapeutic proteins through the combination of three process operations which will contribute to eliminate the immunogenic protein aggregates and block their spontaneous regeneration with time. It is recalled that conventional methods for terminating such production process goes through diafiltration for buffer exchange, optional storage of the drug substance, addition of last components, conventional filtration and dispensation of the drug product into vials.

In contrast, according to some embodiments of the method described herein, after diafiltration and potential addition of the last media components, a new termination of the production process is performed which proceeds through the following steps:

a) full solubilization of the therapeutic protein in a specific final formulation medium,

b) a nanofiltration of the solubilized protein in said medium, optionally adjusting the dilution of the protein in the same buffer, preferably followed by sterile filtration (0.22 μm) and immediate dispensation in the pharmaceutical containers (vials, syringes, sterile pouches . . . ), and

c) optionally, a freeze drying of the nanofiltrate.

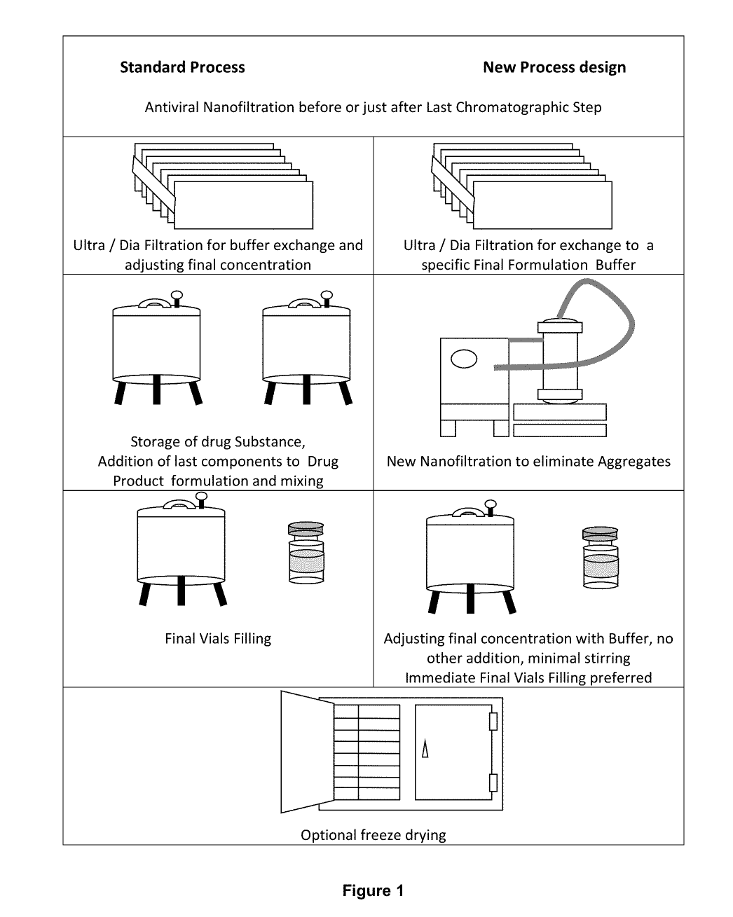

The embodiment of the method which is described above is illustrated in FIG. 1.

It is essential for performing the above embodiment of the method according to the invention to quickly implement these three consecutive steps as the very last steps of a method for preparing a pharmaceutical composition comprising one or more protein active ingredient(s). The absence of significant process step following the nanofiltration step will avoid further stressing of the one or more protein active ingredient(s) before dispensing the composition into the appropriate pharmaceutical containers. In embodiments wherein the resulting nanofiltrate is stored before dispensation into the final pharmaceutical containers, the said nanofiltrate is preferably stored frozen.

Preferred Embodiments of the Starting Composition to be Subjected to Nanofiltration

Prior to the nanofiltration, the therapeutic protein shall preferably be purified to a level that will satisfy all quality attributes defined in the standard regulatory file of a pharmaceutical biotech product. Usually at this stage, the purified protein is in a buffer compatible with the optimal performance of the last purification step, very often a last chromatographic step like ion exchange, gel permeation or hydrophobic interaction chromatography.

In preferred embodiments of the method according to the present invention, the initial buffer resulting from the last process step is changed, so as to place the protein in a specific medium which will favor the removal of the non-covalent aggregates during the nanofiltration and will later limit their spontaneous regeneration with time. In most cases this change of medium will be achieved by performing a diafiltration step which immediately precedes the nanofiltration step. This means that, as disclosed in the present specification, the final nanofiltration step is preferably performed on the final buffered formulation of the protein composition, thus after the step of diafiltration. The final nanofiltration step may preferably be followed by (i) an optional adjustment of the protein concentration and by (ii) an immediate dispensation of the resulting pharmaceutical composition into pharmaceutical containers. Then the dispensed drug product could optionally be freeze dried.

Ideally in the present invention this specific medium should be a pharmaceutically acceptable carrier buffered at a pH distant from the isoelectric point of the protein to preserve its electric charge. For a non-glycosylated protein, the isoelectric point is preferably determined by isoelectric focusing, either by gel or by capillary electrophoresis techniques. For a glycosylated protein with various glycoforms, each glycoform has a specific isoelectric point, so it is important to determine the average isoelectric point weighted for the amount of each of these glycoforms. The use of two-dimension gel electrophoresis or any equivalent capillary electrophoresis method may be helpful to determine this weighted average isoelectric point of a glycoprotein.

Once the isoelectric point of the protein is determined, which is a standard practice in biotech production, the intermediate composition to be nanofiltered is preferably buffered at a pH distant by at least 0.2 pH units from the isoelectric point (pI) of the protein, or the weighted average pI of the various glycoforms, preferably but not exclusively to the acidic side (low pH). This will preserve the electric charge of the protein and accordingly its solubility. Monoclonal antibodies with high pI (8 to 9) can easily be buffered between pH 6 and pH 8.

Some buffers like acetate, citrate, tris, histidine are preferred because they are known as more stabilizing and will prevent pH shift during the lyophilization cycle.

The final adjustment of the pH of the pharmaceutical composition ready for nanofiltration will be adjusted to optimize the solubilization of the protein and also according to laboratory testing of the nanofiltration process in order to optimize the flowing of the protein composition while removing the aggregates.

In some preferred embodiments of the method, the specific buffer for final formulation of the protein composition ready for nanofiltration will include two opposite charged amino acids, an acidic and a basic, such as glutamate and arginine. Although these two amino acids appear optimal and are preferred in the present invention, glutamate could also be substituted for by aspartate or any acidic synthetic analog of these natural amino acids. Although arginine is highly preferred it could also be substituted for by lysine or histidine or any basic synthetic analog of these natural amino acids. The function of these opposite charged amino acids is to mask the protein hydrophobic surfaces/patches. The effective charge on the surface of the protein molecule has significant impact on its colloidal stability by promoting molecular electrostatic repulsion, thereby limiting aggregation. On the opposite, addition of hydrophobic amino acids in the nanofiltration buffer at the antiviral nanofiltration step will improve viral elimination by sticking to the viral particles and increasing their apparent sizes.

The molarity of these opposite charged amino acids in the final formulation of the protein composition is preferably in the 10 mM range, typically ranging from 10 mM to 200 mM each, preferably 20 mM to 60 mM each, most preferably close to 50 mM each. Inside this molarity range, lower figures will be preferred for low molecular weight proteins or proteins with a low frequency of charged amino acids in their primary sequence, while higher figures will be preferred for high molecular weight proteins or proteins with a high frequency of charged amino acids in their primary sequence. The molar concentration of the acidic amino acids and of the basic amino acids, respectively, is preferably in the same range, with the molar ratio of the acidic amino acid over the basic amino acid preferably varying from 0.3 to 3.

The optimal molarity of the opposite charged amino acids (such as arginine and glutamate), is preferably adjusted at laboratory scale to improve the solubilization of the protein and also by measuring the residual content of the protein composition in non-covalent aggregates after the nanofiltration and during the stability studies. These routine operations of stability assessment are preferably performed on either the liquid formulation or on the freeze dried formulation after reconstitution with diluent (like USP water for injection WFI). Various techniques are available for quantifying the residual presence of non-covalent aggregates among which micro flow imaging appears the most convenient and reliable.

The measure of the protein osmotic second virial coefficient (also called B22) by either static light scattering or self-interaction chromatography may advantageously be used for the optimization of the colloidal stability of the protein solution. Adjusting pH, ionic strength and molarity of the components by measuring the second virial coefficient may favor intermolecular electrostatic repulsion and thus prevent the regeneration of aggregates. Assessment of colloidal stability at the lowest ionic strength will be particularly effective for the development of protein formulations of the present invention. Optimizing the colloidal stability by measuring the second virial coefficient will lead to decreasing the ionic strength to preserve the net charge of the protein packed with the charged amino-acids. Using low molarities of salt, such as a NaCl concentration lower than 100 mM, is believed to be advantageous for performing the method for preparing a pharmaceutical composition that is described herein.

In some embodiments, one or more compounds may be added to the starting composition to be nanofiltered, such as surfactants, antioxidants and antimicrobial preservatives.

Addition of surfactants like Polysorbate 20 or 80 (Tween 20 or 80) could be considered because they can prevent the formation of non-covalent aggregates by interaction of the proteins with foreign materials such as glass or rubber particles or droplets of silicone oil. These aggregates, usually of large size (3 to 30 m are common) also have an immunogenic potential. Besides the addition of Polysorbate limit the regeneration of aggregates produced by shaking during the handling and shipping of the drug product.

Addition of antioxidants like methionine or reduced glutathion could also be considered because they can prevent the oxidation of the protein. Oxidized forms of the proteins are subject to aggregation and in turn can increase the risk of production of new aggregates through the process of nucleation.

In some embodiments, antimicrobial preservatives may be added before adding any antimicrobial preservatives such as those used for multi-use vials or containers, their ability to trigger the generation of aggregates should be carefully checked at lab scale. Benzyl alcohol is an inducer of protein aggregates and should definitively be excluded from protein compositions of the present invention (Zhang et al., 2004, J. Pharm. Sci. 93, 3076-3089; Roy et al., 2005, J. Pharm. Sci. 94, 382-396; Thirumangalathu et al., 2006, J. Pharm. Sci. 95, 1480-1497).

The conditions, which include the pH, addition of amino acids or other pharmaceutically acceptable substances and other conditions as described herein, are chosen so as to optimize the solubilization of the protein, dissociate soluble aggregates while not inducing further re-aggregation of the protein after nanofiltration. In that aim the adjustment of the pH at distance from the protein pI and the addition of the tandem opposite charged amino-acids are critical. This minimizes or eliminates the soluble aggregates of the protein and therefore improves the quality of the protein therapeutic. Adjustment of these conditions should be tested at laboratory or pilot scale before finalizing the formulation of the protein composition.

Preferred Embodiments of the Nanofiltration Step

Conventionally, nanofiltration of glycosylated recombinant proteins is often used in the art to eliminate potential viral contaminants. This viral elimination step usually occurs during the purification of the protein in a buffer compatible with process operations and virus removal, at the latest before the ultrafiltration/diafiltration used for buffer exchange. It delivers a drug substance devoid of viral contaminants (Liu et al., 2010, mAbs 2, 480-499; table 2 page 496).

In contrast, the primary function of the final nanofiltration step of the present invention's method does not consist of eliminating viral contaminants, although this final nanofiltration step may also partly contribute to viral clearance in the overall protein purification process.

As described throughout the present specification, it is key to perform the nanofiltration step of the invention's method as the ultimate step or quasi-ultimate step of the process, in the final formulation of the protein composition, thus after a process step aimed at placing the protein in its final formulation form, and preferably just before dispensing the protein composition into the vials, syringes or any other pharmaceutical container, ready for liquid storage or subsequent freeze drying.

Thus the method according to the invention discloses a new way of using the nanofiltration devices, which is to eliminate the soluble covalent and non-covalent micrometric and sub-micrometric aggregates of therapeutic protein-containing compositions.

It is also disclosed herein optimal ways to operate the final nanofiltration step for the proper elimination of these protein aggregates. In preferred embodiments, this involves the use of media, typically buffer solutions, which are distinct from the conventional buffer solutions used to optimize the viral elimination.

The performance of a standard antiviral nanofiltration step may be assessed on the ability of this step to clear the virus used during the viral spiking tests. In contrast, the performance of the final nanofiltration step that is performed according to the invention's method is assessed on its ability to reduce the content of the protein composition in soluble protein aggregates. Specific analytical methods among which microflow imaging are preferably used for assessing the performance of the final nanofiltration step of the invention's method.

It is recalled that the nanofiltration step of the method according to the invention is performed at the very end of a method for preparing a pharmaceutical composition, preferably just before the dispensation of the protein composition in vials or syringes or any type of container conventionally used in the pharmaceutical industry. Accordingly this nanofiltration step is preferably either the last process step performed for the production of the “drug substance” or included in the production of the “drug product”. Here the terms “drug substance” and “drug product” are used according to their pharmaceutical definitions as referred to in the International Conference Harmonization guidelines for biotech productions.

For performing the nanofiltration step of the invention's method, it may be used any kind industrial device that is conventionally utilized for performing an “antiviral” nanofiltration step. Some suitable filters having the required porosity for performing the final nanofiltration step of the invention's method may be selected in a group comprising hollow fiber filters containing a bundle of straw-shaped hollow fibers. The wall of each hollow fiber has a three-dimensional web structure of pores comprised of voids interconnected by fine capillaries. Such is for instance the Asahi-Kasei “Planova™” device. Other filters are made of dual layers synthetic membranes (PVDF or PES), such is the “Viresolv Pro™” device from Merck-Milllipore, the Pall Ultipor and the Sartorius Virosart. (Liu et al., 2010; mAbs 2, 480-499, table 1 page 492).

The porosity of the filter is preferably in the ten nanometer range. Porosities of 20 nm to 30 nm are preferred. Laboratory pretests will determine the optimal choice of porosity to optimize the removal of the aggregates and preserve a reasonable process flow and protein recovery during the nanofiltration. This will also be adjusted to the average molecular weight of the protein to insure the perfect flowing of the protein monomer through the filter. Filter manufacturers provide guides to adjust the porosity of the hollow fiber filter to the molecular weight of the protein. A cytokine can be nanofiltered with a filter porosity of 15 nm to 30 nm, while larger molecules like antibodies or recombinant factor VIII might require a porosity of 40 nM to deliver an acceptable process flow. The manufacturer of these devices propose various scales to evaluate the average porosity of the filters, these are expressed according to the molecular weight (expressed in kilodaltons) of the proteins to be submitted to nanofiltration or to the average size in nanometers of the viral particles retained by the filter. To avoid fouling the solution can be pre-filtered on a 0.1 μm or 0.2 μm filter.

Preferred Embodiments of the Optional Final Steps of the Method

Once the finally formulated protein nanofiltered composition is obtained, the resulting nanofiltrate is preferably quickly dispensed into the vials or any other container of pharmaceutical use. Then, said vials or containers may optionally be submitted to a freeze drying step in order to ensure a long term stability. Should the nano-filtrated protein composition be stored for a few days before filling the final containers (vials, syringes . . . ), then it's highly preferable to freeze the nanofiltrate for this limited storage period but this freeze thaw operation should be limited to one freeze-thaw cycle. The operation of freeze drying will contribute to prevent the regeneration of non-covalent aggregates and generally stabilize the protein composition during long term storage.

Standard cycle of freeze drying may be adapted to each protein formulation with the aim of minimizing the regeneration of protein aggregates. Thermal analysis of the protein composition will determine the glass transition temperature and collapse temperature. For the purpose of the present invention, conducting the freezing step to bring the temperature below the glass transition or the collapse temperature is not an absolute requirement. The minimization of aggregates should govern the design of the lyophilization cycle and its optimization, whatever the visual appearance of the cake.

The production of a cake with an elegant visual aspect is often looked for in the pharmaceutical industry. This is obtained by addition of cryoprotecting agents such as polyols (mannitol, sorbitol) or sugars (fructose, threalose, etc). In the present invention it is preferable to avoid the polyols (mannitol, sorbitol) these agents can contribute to the immunogenicity of the protein composition. The elegant appearance of the cake after freeze drying does not contribute to the stability of the protein composition. In the present invention the visual aspect of the cake should not be retained as a decisive parameter for batch release of the pharmaceutical composition. Cake collapse after freeze drying should be accepted and the standard use of polyols or sugars will be advantageously substituted for by addition of the charged amino acids previously mentioned.

Optionally the formulation of the protein composition ready for nanofiltration will be completed by addition of neutral amino acids like glycine, alanine, leucine, or hydroxylated amino acids like serine or threonine to ballast the medium for freeze drying, but the tandem addition of arginine and glutamate remains the preferred choice in the present invention and their molarities should remain higher than the molarity of the neutral amino acids.

The protein composition could be dispensed into vials, cartridges or syringes before freeze drying. In such case contamination of the protein composition by droplets of silicone oil should be monitored and avoided. The use of amber vials will be preferred to protect from oxidation due to light exposure.

The reconstitution of the freeze dried protein composition will be performed just before administration to the patient with a pharmaceutically acceptable diluent. The ionic strength of the diluent will be established to approach isotonicity. Nevertheless preserving the colloidal stability of the reconstituted product with a low ionic strength should be privileged even at a price of a moderate hypotonicity. In a preferred embodiment the protein composition adjusted by dilution after nanofiltration, will have a ionic strength allowing reconstitution of the freeze dried product with sterile water for injection (USP WFI).

The administration of the final protein composition to the patient should preferably be performed by intra-muscular or intra-venous route or by mucosal delivery. The subcutaneous and intra dermal routes being more immunogenic they should be avoided for the administration of the protein composition of the present invention.

Pharmaceutical Compositions

The present invention also relates to a pharmaceutical composition that is obtained by performing the invention's method described herein.

Notably, this invention pertains to a pharmaceutical composition having a reduced content in subvisible micrometric protein aggregates of a size ranging from 0.1 μm to 50 μm.

In some embodiments of the pharmaceutical composition, the concentration of large micrometric protein aggregates (3 to 30 μm in size) detected by microflow imaging is reduced by at least 75% in comparison to the same composition not treated by nanofiltration.

In some embodiments of the pharmaceutical composition, the concentration of large micrometric protein aggregates (3 to 30 μm in size) detected by microflow imaging remains reduced by 70% when stored at 4° C. for one month.

In some embodiments of the pharmaceutical composition, the protein is solubilized in a pharmaceutically acceptable carrier buffered at least at 0.2 pH units, preferably at least minus 0.2 pH units, from the isoelectric point of the therapeutic protein or from the weighted average isoelectric point of the various glycoforms of said protein in said composition.

In some embodiments of the pharmaceutical composition, the protein is solubilized in a pharmaceutically acceptable carrier having a pH value selected in a group comprising (i) a pH value of 0.2 pH units or more higher than the isoelectric point of the said protein active ingredient, and (ii) a pH value of 0.2 pH units or less lower than the isoelectric point of the said protein active ingredient.

In some embodiments of the pharmaceutical composition, the protein is solubilized in a pharmaceutically acceptable carrier having a pH value of 0.2 pH units or less lower than the isoelectric point of the said protein active ingredient.

In some embodiments of pharmaceutical composition, the pharmaceutically acceptable carrier comprises at least two oppositely charged amino acids, at least one acidic and one basic, preferably arginine and glutamate.

In some embodiments of the pharmaceutical composition, the basic amino-acid is chosen among arginine, lysine, histidine or their various charged synthetic analogs and the acidic amino-acid is chosen among aspartate, glutamate or their various charged synthetic analogs.

In some embodiments of the pharmaceutical composition, all charged amino-acids are present at a total molarity of 20 to 200 mM, preferably 50 to 100 mM.

In some embodiments of the pharmaceutical composition, the molarity ratio of the acidic over basic amino acid is comprised between 0.3 and 3.

In some embodiments of the pharmaceutical composition, the pharmaceutically acceptable carrier also contains neutral amino-acids like glycine, alanine, leucine or isoleucine, and/or hydroxyl amino-acids like serine or threonine, the total molarity of which remaining below the molarity of the charged amino acids.

In some embodiments of the pharmaceutical composition, the pharmaceutically acceptable carrier also contains a surfactant agent like Polysorbate 20 or 80.

In some embodiments of the pharmaceutical composition, the protein is endogenous to the species of the individual.

In some embodiments of the pharmaceutical composition, the protein is a cytokine. In some embodiments, the cytokine is selected in a group comprising an interleukin, which encompasses IL-7, IL-2, IL-21, IL-15 and IL-12. In some embodiments, the cytokine is selected in a group comprising an interferon, which encompasses interferons α, β, δ, γ, λ and their close analogs.

In some embodiments of the pharmaceutical composition, the protein is a fusion protein comprising a cytokine or the soluble receptor of a cytokine (interleukin or interferon) and the Fc fragment of an immunoglobulin.

In some embodiments of the pharmaceutical composition, the protein is an immuno-activating monoclonal antibody like anti-PD1, anti-PDL1, anti-CTLA-4, anti-Lag3, anti-Tim3, anti-TGFβ.

In some embodiments of the pharmaceutical composition, the recombinant protein is a hormone, a growth factor or an enzyme used for chronic therapy.

In some embodiments of the pharmaceutical composition, the recombinant protein is a human growth hormone or an anti-hemophilic factor like factor VII or VIII.

Methods for detecting and quantifying protein aggregates Several methods are available for analyzing and quantifying aggregated proteins.

The standard method for detecting protein aggregates smaller than 0.1 micron is size exclusion chromatography or gel permeation chromatography. The usual method used to detect protein aggregates and particles larger than 20 micrometers is the USP light obscuration technique. Other techniques are available but these two approaches represent the most common practices for a man skilled in the art. Although it is now well recognized by industry and regulatory agencies that protein aggregation is a main factor causing therapeutic protein immunogenicity some aggregates in the 0.1 μm to 10 μm range still go undetected, in-part due to the conventionally accepted analytical techniques. This gap in the detection of protein aggregates explains their presence in many approved commercial products.

Among the less classical but more recent techniques useful for the detection and quantification of protein aggregates we can quote: Analytical ultracentrifugation (through the measure of sedimentation velocity) (Philo, 2009, Curr. Pharm. Biotechnol. 10, 359-372), Asymetrical Field flow fractionation (Hawe et al., 2012, J. Pharm. Sci. 101, 4129-4139), Light scattering methods, static or dynamic, such as methods using laser light scattering (Arakawa and Philo, 2007, Aggregation Analysis of Therapeutic Proteins, Part 2. Bioprocess Int. 36-47) (Nobbmann et al., 2007, Biotechnol. Genet. Eng. Rev. 24, 117-128), Nanoparticles tracking analysis (Nanosight Ltd) where samples are illuminated by a laser and particle movement is tracked via light scattering by a CCD camera (Filipe et al., 2010, Pharm. Res. 27, 796-810). Other general techniques are described in US Patent Application Publication No. 2008/0161242 and 2012/0070406 and were extensively reviewed and updated by Zölls et al., in 2012 (Particles in therapeutic protein formulations, Part 1: overview of analytical methods. J. Pharm. Sci. 101, 914-935).

The recent development of micro-flow imaging (MFI) provides a new technology for measuring the number and size of sub-visible particles in a solution. This technology can assess aggregates in the micrometer size range. During micro-flow imaging, digital microscopy images of a protein solution are taken relative to a blank, and aggregate content is measured by quantifying the size and number of particles present. Apparatus for micro-flow imaging of particles are commercially available from Brightwell Technologies, Inc. (Protein Simple) and Occhio Belgium (like the flowcell FC200S used in the examples of the present invention).

In the present invention, micro-flow imaging (MFI) (Sharma et al., 2010a, AAPS J. 12, 455-464) (Sharma et al., 2010b, J. Pharm. Sci. 99, 2628-2642) appeared the most reliable technology to evaluate particle numbers and particle sizes of protein samples, particularly in the subvisible range known to be source of immunogenicity (e.g., about 0.2 to about 30 microns in size). The presence and/or level of such subvisible particles is indicative of an immunogenic preparation. Moreover a shift from the low size particles 0.4μ to the high size particles 5 to 301μ directly associates with the presence of protein aggregates in the composition tested. The Micro-flow imaging method has been used in the examples herein for detecting and quantifying protein aggregates.

For the present invention we called “micrometric aggregates”: protein/protein aggregates or protein/foreign particles aggregates with a size comprised between 0.2 m and 50 μm. These aggregates are detected by micro flow imaging technologies.

As now well recognized in the literature, subvisible protein particles made of non-covalent protein aggregates, at levels undetectable by standard analytical methods such as size exclusion chromatography and light obscuration can induce immune responses to a self protein or epitope. Specifically, aggregates detected by MFI, which could not previously be detected by SEC-HPLC as they were below the limit of detection or not eluted from the column, can have significant immunogenic potential (Marszal and Fowler, 2012, J. Pharm. Sci. 101, 3555-3559). As stated above a shift to higher size in particles size distribution increases the risk of immunogenicity.

### Examples

### Example 1—Detection of Protein Aggregates by Micro-Flow Imaging

A DPA-4100 particle analyzer system (ProteinSimple, Santa Clara, USA) equipped with a high-resolution 100 μl flow cell can be used. Samples are analyzed without any dilution, but usually tested at 1 mg/mL. A pre-run volume of 0.3 ml is followed by a sample run of 0.65 ml. Approximately 1100 images can be taken per sample. Between the measurements, the flow cell is cleaned with purified water. Results are analyzed using the MFI view analysis suite software. Size distribution, aspect ratio and illumination intensity level are analyzed.

An Occhio Micro Flow Imaging (Occhio Flow Cell FC200S) system was used to produce the data provided here as examples. This orthogonal method allows the analysis of protein aggregation mainly in the range of 200 nm up to 1000 μm. 300 μl of the sample were analyzed after prior dilution to bring the protein concentration down to 1 mg/ml (except for beta interferon). The high resolution camera allows the collection of images and direct counting of particles. The device also provides information related to the size and shape of the particles.

Typically in our various experiments, we were able to detect a few hundreds to a few ten thousands of particles per ml of protein solution. Their sizes ranged approximately from 0.2 μm to 50 μm and the device allows to produce an histogram of the distribution of particles sizes. In the following examples we have pooled the particles sizes to evaluate their distribution over three significant classes of aggregates: <1 μm, 1 to 5 μm, >5 μm.

This analytical technology was used to optimize the best solution buffer, to demonstrate the effect of applying nanofiltration as the ultimate process step just before vialing and optionally freeze drying. It was also used to explore the effect of various stress like shaking, heating, freezing and thawing the pharmaceutical compositions, optionally followed by nanofiltration.

### Example 2—Methods for Determining Immunogenicity

The MSD bridging immunogenicity assay is used to detect and quantify antibodies. This method is more sensitive than the usual method used (sandwich ELISA). The MSD technology is based on electrochemiluminescence detection principle and uses streptavidin-coated microplates with electrodes integrated into the bottom of the plate. Therapeutic proteins were combined to Sulfo-tag on one hand and to biotin molecules on the other hand. The anti-therapeutic proteins binding antibodies are recognized by these two combined proteins reagents as follows: the therapeutic proteins labeled molecules are used in a two-site sandwich format including both the coating (proteins labeled with biotin) and the detector (proteins labeled with sulfo-tag).

If binding antibodies have been detected in a sample, their neutralizing potential against therapeutic proteins is detected by using a functional assay like cell-based bioassays or enzyme assays. Most of these assays are periodically reviewed by the WHO experts committee in biological standardization.

### Example 3—Nanofiltration of Solubilized Therapeutic Protein Compositions

Most therapeutic protein compositions have a protein concentration varying between 0.1 mg/ml to 30 mg/ml.

Such protein concentrations can easily be handled by the Asahi Kasei Planova filters, using the 15N type for the smaller molecules and lower concentrations and the 20N or the BioEX for the larger molecules and higher protein concentrations. The loss of protein due to nanofiltration is low <10% (Asahi Kasei). A pre-filtration with standard 0.1 μm or 0.22 μm filters can easily block the potential fouling effect upstream from the nano-filtering device.

For the purpose of antiviral treatment, the efficient nanofiltration of various proteins has already been documented with these Planova filters:

growth factors like G-CSF, Interleukins, Erythropoietin,

Coagulation factors like Factor VII, VIII, IX, XI, vWF

therapeutic enzymes like □1 antitrypsin, antithrombin III, tPA

various monoclonal IgGs

In the following examples, due to the scarcity of the samples we used the smallest nano-filter sizes 0.001 or 0.0003 m².

### Example 4—Reduction of Aggregates in an Experimental Monoclonal Antibody

A generic IgG monoclonal antibody “A” was diafiltered with a macrosep centrifugal device (Pall Corporation) and a 30K omega membrane to be re-solubilized in a 50 mM acetate buffer pH 5.5 complemented with 50 mM L-Arginine, 50 mM L-Glutamic Acid, 10 mM glycine, 50 mM NaCl and 0.02% polysorbate 80. The concentration of the bulk IgG was 10 mg/mL.

The solubilized “A” antibody was then submitted to two different stress: stirring during 3 consecutive days or 4 subsequent freeze thaw cycles. Meanwhile another sample was filtered through a standard 0.22μ filter and then nanofiltered through a PLANOVA 20N. The nanofilter was washed with the same buffer.

After dilution of the antibody to 1 mg/mL in the same buffer, various sets of measurements were performed by MFI to quantify the aggregates in three size classes <1 μm, 1 to 5 μm, >5 μm.

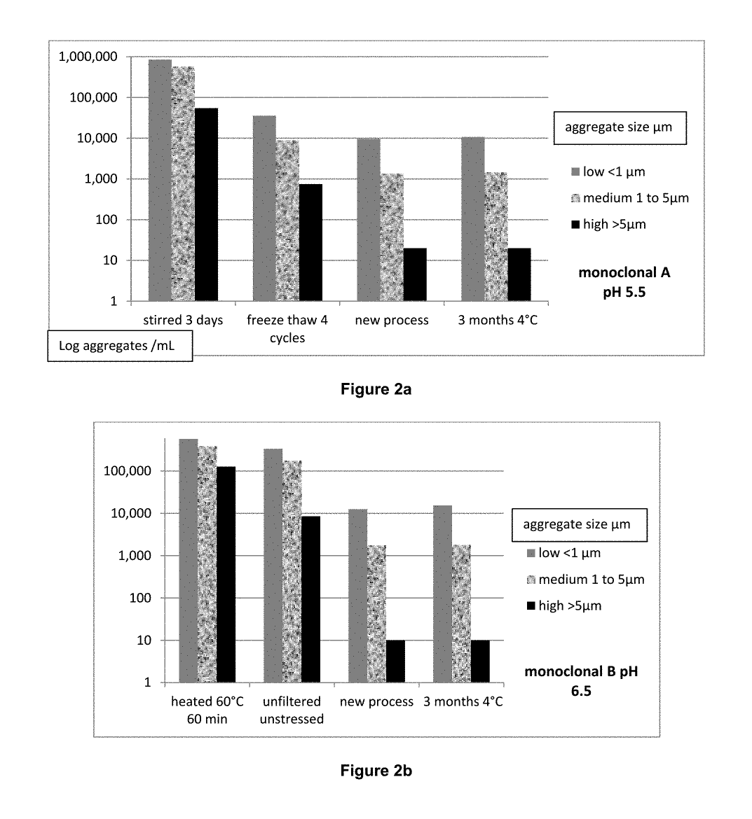

The data representing the means of 5 sets of measures with the Occhio Flow Cell FC200S⁺ are presented in FIG. 2a showing the large aggregate contents of stirred and frozen thawed samples, while the nanofiltered samples only contain low amounts of micrometric aggregates.

Another experimental IgG monoclonal antibody “B” was diafiltered with a macrosep centrifugal device (Pall Corporation) and a 30K omega membrane to be re-solubilized in a 25 mM citrate buffer pH 6.5 complemented with 50 mM L-Arginine, 50 mM L-Glutamic Acid, 10 mM glycine, 80 mM NaCl. The antibody was then heated 1 hour at 65° C. and nanofiltered on a PLANOVA 20N. Samples of the heated antibody before or after nanofiltration were diluted to 1 mg/ml in the same buffer and aggregates were measured by MFI.

The data representing the means of 4 sets of measures performed with the Occhio Flow Cell FC200S⁺ are presented in FIG. 2b.

### Example 5—Reduction of Interleukin-7 Aggregates to Produce a Non Immunogenic IL-7 Pharmaceutical Preparation

The expression of non-glycosylated Interleukin-7 is conducted by culturing a recombinant E Coli clone, while glycosylated Interleukin-7 is expressed from a mammalian cell clone (CHO), both bearing the IL-7 gene sequence and appropriate coding regions to promote IL-7 gene expression and IL-7 protein secretion in the culture medium.

Crude culture medium is collected and purified according to standard purification methods including various filtration, ion exchange chromatographies, finishing by a “polishing” step based on hydrophobic interaction chromatography (HIC). This is best exemplified in U.S. Pat. No. 7,585,947 and U.S. Pat. No. 8,034,327.

The Filtered Pooled HIC eluates is then concentrated and exchanged with 6 diavolumes of diafiltration buffer: 20 mM Sodium Acetate, 60 mM NaCl, 50 mM L-Arginine, 50 mM L-Glutamic Acid, pH 5.0. A wash/recirculation is performed to recover the Diafiltered Retentate. The Recovered Retentate is then diluted with the same buffer to the desired concentration, usually 2 to 4 mg/mL. This sterile solution is filtered through a 0.22 μm filter and collected into a sterile bioprocess container. At this first step of the invention, the protein and non-covalent aggregates are solubilized in the pH 5 arginine/glutamate Na acetate buffer.

To execute the second step, the solution is prefiltered through a 0.221 μm pre-filter, and then nanofiltered through a Planova 20N virus removal filter. A wash is performed with 20 mM Sodium Acetate, 60 mM NaCl, 50 mM L-Arginine, 50 mM L-Glutamic Acid, pH 5.0 to recover the product. The Recovered Nanofiltrate is diluted to the desired concentration with the same buffer, usually 2 to 4 mg/mL.

The Diluted Nanofiltrate contains very low amounts of these contaminants. It is 0.22 μm sterile filtered and bulk filled before storage at −20° C. or immediately dispensed in the vials chosen for pharmaceutical use before being freeze dried and appropriately labeled. Amber borosilicate glass vials are adequate for the storage of the freeze dried product and preserve the lyophilized IL-7 from light oxidation.

The diluted nanofiltrate was also tested after short term (1 to 3 months) storage at 4° C. showing a non significant regeneration of aggregates.

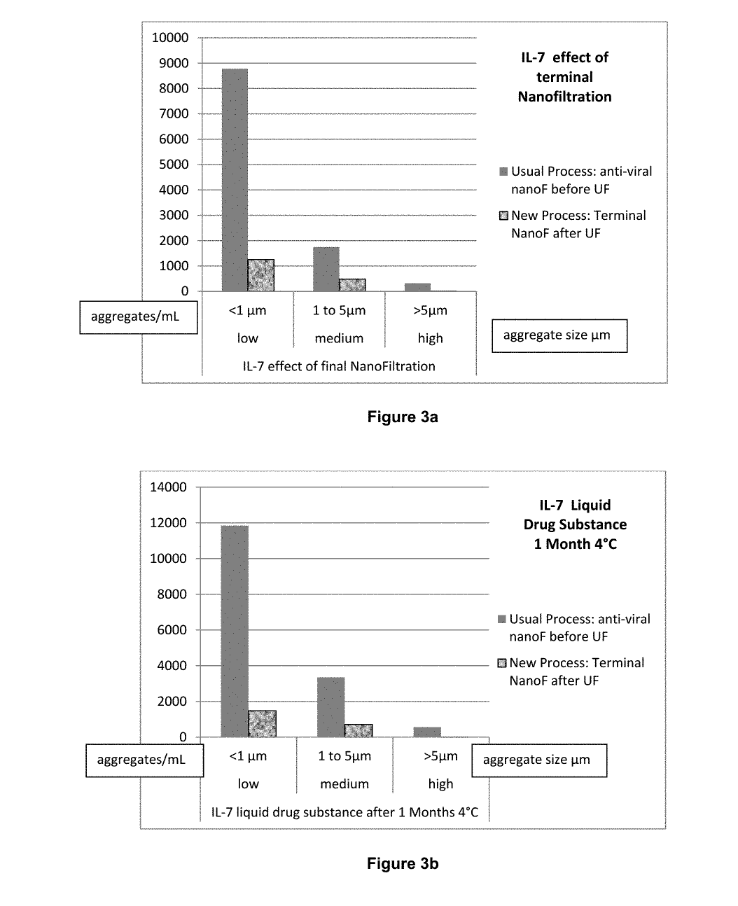

FIGS. 3a and 3b provides examples of IL-7 aggregates reduction with this procedure

### Example 6—Reduction of Aggregates in an Experimental Preparation of Factor VIII

A sample of a commercial source of a purified freeze dried factor VIII is diluted with water (USP WFI) to a concentration of 100 I.U./mL (approximately 25 μg/mL) and diafiltered with a microsep centrifugal device (Pall Corporation) and a 100K omega membrane against the following buffer. 40 mM L-Arginine, 60 mM L-Glutamic Acid, 10 mM glycine, 20 mM histidine, 60 mM NaCl, 4 mM CaCl₂ and 0.02% polysorbate 80.

The sample is then filtered through a standard 0.22μ filter and then nanofiltered through a PLANOVA BioEX lab scale (0.0003 m²). The nano-filtrate is immediately freeze dried in this buffer.

Another sample of a commercial source of a purified freeze dried factor VIII is diluted with water (USP WFI) to a concentration of 100 I.U./mL (approximately 25 μg/mL) and diafiltered with a microsep centrifugal device (Pall Corporation) and a 100K omega membrane against the following buffer: 40 mM L-Arginine, 60 mM L-Glutamic Acid, 30 mM sucrose, 20 mM histidine, 60 mM NaCl, 4 mM CaCl₂ and 0.02% polysorbate 80.

The sample is then filtered through a standard 0.22μ filter and then nanofiltered through a PLANOVA BioEX lab scale (0.0003 m²). The nanofilter is washed with the same buffer and protein concentration adjusted to the desired concentration. The nano-filtrate is immediately freeze dried in this buffer.

We also successfully tested the following formulation: 40 mM L-Arginine, 60 mM L-Glutamic Acid, 30 mM Trehalose, 20 mM histidine, 60 mM NaCl, 4 mM CaCl₂ and 0.02% polysorbate 80.

### Example 7—Reduction of Aggregates in an Experimental Preparation of Beta Interferon

A sample of a commercial source of human beta interferon was diluted with water to a concentration of 250 μg/ml and diafiltered with a microsep centrifugal device (Pall Corporation) and a 10K omega membrane against the following buffer: 40 mM acetate buffer pH 5.5 complemented with 50 mM L-Arginine, 50 mM L-Glutamic Acid, 10 mM glycine, 40 mM NaCl, and 0.005% polysorbate 20. The protein concentration was set at approximately 0.5 mg/mL.

The sample was then filtered through a presoaked standard 0.22 μm filter and nanofiltered through a PLANOVA BioEX lab scale (0.0003 m²). After washing the nanofilter with the same buffer and adjustment of protein concentration to 0.25 mg/mL the samples were analyzed by MFI.

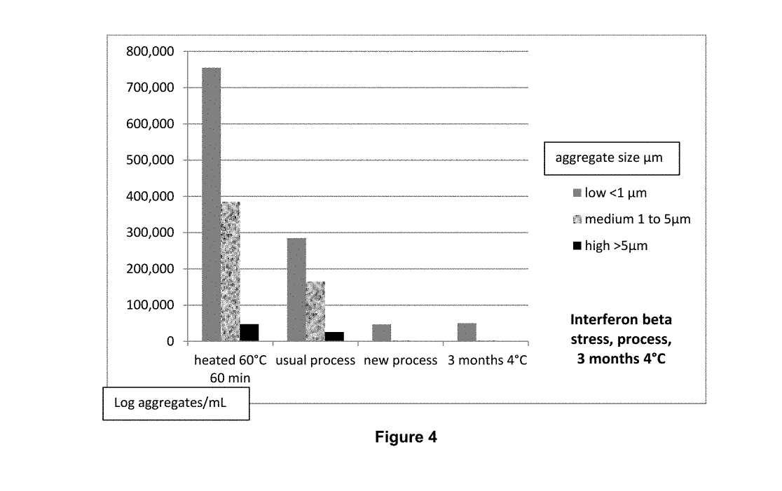

The data representing the means of 3 sets of measures made with the Occhio Flow Cell FC200S⁺ are presented in FIG. 4.

### CONCLUSIONS

The present invention discloses stable protein compositions with reduced protein aggregates. They will be useful for the therapy of patients at risk of generating antidrug antibodies. Such compositions will be advantageously used for therapeutic proteins or monoclonal antibodies aimed at stimulating the immune system and for therapeutic proteins used in chronic therapies. These are two therapeutic situations known to favor the generation of anti-drug antibodies.

These protein compositions have been nanofiltered in their final formulation which has been specifically designed to preserve the electric charge of said proteins in said compositions. Such compositions have a very significantly reduced content in protein aggregates. Moreover after storage at 4° C. they demonstrate their stability, showing only a non significant regeneration of such aggregates.

The invention discloses the specific technology used to prepare such compositions which mainly includes a terminal nanofiltration in a specific buffer containing a set of opposite charged amino acids. Contrary to the common use of nanofiltration to eliminate viral particles, this technology should be implemented in the very final formulation of said protein composition. This technology is easy to use at industrial scale and produces a drug substance ready for dispensation into pharmaceutical containers and optional freeze drying.

### REFERENCES

Arakawa, T., and Philo, J. S. (2007). Aggregation Analysis of Therapeutic Proteins, Part 2. Bioprocess Int. 36-47.
Asahi Kasei Asahi Kasei Planova Brochure.
Barnard, J. G., Singh, S., Randolph, T. W., and Carpenter, J. F. (2011). Subvisible particle counting provides a sensitive method of detecting and quantifying aggregation of monoclonal antibody caused by freeze-thawing: insights into the roles of particles in the protein aggregation pathway. J. Pharm. Sci. 100, 492-503.
Bee, J. S., Goletz, T. J., and Ragheb, J. A. (2012). The future of protein particle characterization and understanding its potential to diminish the immunogenicity of biopharmaceuticals: a shared perspective. J. Pharm. Sci. 101, 3580-3585.
Carpenter, J., Cherney, B., Lubinecki, A., Ma, S., Marszal, E., Mire-Sluis, A., Nikolai, T., Novak, J., Ragheb, J., and Simak, J. (2010). Meeting report on protein particles and immunogenicity of therapeutic proteins: filling in the gaps in risk evaluation and mitigation. Biol. J. Int. Assoc. Biol. Stand. 38, 602-611.
Cleland, J. (2013). USP 0130058895 High Pressure Treatment of Aggregated Interferons.
Den Engelsman, J., Garidel, P., Smulders, R., Koll, H., Smith, B., Bassarab, S., Seidl, A., Hainzl, O., and Jiskoot, W. (2011). Strategies for the assessment of protein aggregates in pharmaceutical biotech product development. Pharm. Res. 28, 920-933.
Filipe, V., Hawe, A., and Jiskoot, W. (2010). Critical evaluation of Nanoparticle Tracking Analysis (NTA) by NanoSight for the measurement of nanoparticles and protein aggregates. Pharm. Res. 27, 796-810.
Hawe, A., Romeijn, S., Filipe, V., and Jiskoot, W. (2012). Asymmetrical flow field-flow fractionation method for the analysis of submicron protein aggregates. J. Pharm. Sci. 101, 4129-4139.
Liu, H. F., Ma, J., Winter, C., and Bayer, R. (2010). Recovery and purification process development for monoclonal antibody production. mAbs 2, 480-499.
Marszal, E., and Fowler, E. (2012). Workshop on predictive science of the immunogenicity aspects of particles in biopharmaceutical products. J. Pharm. Sci. 101, 3555-3559.
Morre, M. (2009). U.S. Pat. No. 7,585,947 IL7 Drug Substance conformere.
Morre, M. (2011). U.S. Pat. No. 8,034,327 Hyperglycosylated IL-7, preparation and uses.
Nobbmann, U., Connah, M., Fish, B., Varley, P., Gee, C., Mulot, S., Chen, J., Zhou, L., Lu, Y., Shen, F., et al. (2007). Dynamic light scattering as a relative tool for assessing the molecular integrity and stability of monoclonal antibodies. Biotechnol. Genet. Eng. Rev. 24, 117-128.
Philo, J. S. (2009). A critical review of methods for size characterization of non-particulate protein aggregates. Curr. Pharm. Biotechnol. 10, 359-372.
Randolph, T. W. (2008). United States Patent Application: 0080161242.
Rosenberg, A. S. (2006). Effects of protein aggregates: an immunologic perspective. AAPS J. 8, E501-507.
Rosenberg, A. S., Verthelyi, D., and Cherney, B. W. (2012). Managing uncertainty: a perspective on risk pertaining to product quality attributes as they bear on immunogenicity of therapeutic proteins. J. Pharm. Sci. 101, 3560-3567.
Roy, S., Jung, R., Kerwin, B. A., Randolph, T. W., and Carpenter, J. F. (2005). Effects of benzyl alcohol on aggregation of recombinant human interleukin-1-receptor antagonist in reconstituted lyophilized formulations. J. Pharm. Sci. 94, 382-396.
Seefeldt, M. (2012). United States Patent Application: 0120070406.
Sharma, D. K., King, D., Oma, P., and Merchant, C. (2010a). Micro-flow imaging: flow microscopy applied to sub-visible particulate analysis in protein formulations. AAPS J. 12, 455-464.
Sharma, D. K., Oma, P., Pollo, M. J., and Sukumar, M. (2010b). Quantification and characterization of subvisible proteinaceous particles in opalescent mAb formulations using micro-flow imaging. J. Pharm. Sci. 99, 2628-2642.
Thirumangalathu, R., Krishnan, S., Brems, D. N., Randolph, T. W., and Carpenter, J. F. (2006). Effects of pH, temperature, and sucrose on benzyl alcohol-induced aggregation of recombinant human granulocyte colony stimulating factor. J. Pharm. Sci. 95, 1480-1497.
Wang, W., Singh, S. K., Li, N., Toler, M. R., King, K. R., and Nema, S. (2012). Immunogenicity of protein aggregates—concerns and realities. Int. J. Pharm. 431, 1-11.
Zhang, Y., Roy, S., Jones, L. S., Krishnan, S., Kerwin, B. A., Chang, B. S., Manning, M. C., Randolph, T. W., and Carpenter, J. F. (2004). Mechanism for benzyl alcohol-induced aggregation of recombinant human interleukin-1 receptor antagonist in aqueous solution. J. Pharm. Sci. 93, 3076-3089.
Zölls, S., Tantipolphan, R., Wiggenhorn, M., Winter, G., Jiskoot, W., Friess, W., and Hawe, A. (2012). Particles in therapeutic protein formulations, Part 1: overview of analytical methods. J. Pharm. Sci. 101, 914-935.

1.-22. (canceled)

23. A method for preparing a pharmaceutical composition comprising a protein active ingredient and having a reduced amount of protein aggregates, the said method comprising performing a step of nanofiltration of a starting composition comprising the said protein active ingredient in a solubilized form, whereby the said pharmaceutical composition is obtained.

24. The method according to claim 23, wherein the protein aggregates are non-covalent immunogenic protein aggregates.

25. The method according to claim 23, wherein the nanofiltration step is performed by using a nanofiltration membrane having a mean pore size of less than 100 nm, advantageously a mean pore size of less than 30 nm, and preferably a mean pore size ranging from 10 nm to 30 nm.

26. The method according to claim 23, wherein the said pharmaceutical composition is introduced in a container after the nanofiltration step; in particular wherein the container is a dispensation container.

27. The method according to claim 23, wherein the pharmaceutical composition is subjected to a step of freeze-drying after the nanofiltration step.

28. The method according to claim 23, wherein, in the said starting composition has a pH value selected in a group consisting of (i) a pH value of 0.2 pH units or more higher than the isoelectric point of the said protein active ingredient, and (ii) a pH value of 0.2 pH units or less lower than the isoelectric point of the said protein active ingredient.

29. The method according to claim 23, wherein, in the said starting composition has a pH value of 0.2 pH units or less lower than the isoelectric point of the said protein active ingredient.

30. The method according to claim 23, wherein the said starting composition comprises at least two amino acids having opposite charges, advantageously one or more basic amino acid and one or more acidic amino acid, and preferably arginine and glutamate.

31. The method according to claim 30, wherein
the one or more basic amino acid is selected in a group consisting of arginine, lysine, histidine and a charged analog thereof; and
the one or more acidic amino acid is selected in a group consisting of aspartate, glutamate and a charged analog thereof.

32. The method according to claim 23, wherein the said at least two amino acids having opposite charges comprise arginine and glutamate.

33. The method according to claim 23, wherein each charged amino acid is present in the intermediate composition at a concentration ranging from 20 mM to 200 mM, and preferably at a concentration ranging from 50 mM to 100 mM.

34. The method according to claim 23, wherein the molar ratio of the acidic amino acid to the basic amino acid ranges from 0.3 to 3.

35. A pharmaceutical composition that is obtained according to the method of claim 23.

36. A pharmaceutical composition comprising one or more proteins as active ingredient(s) having a reduced content in micrometric protein aggregates of a size ranging from 0.1 μm to 50 μm.

37. The pharmaceutical composition according to claim 36; wherein the protein aggregates are non-covalent immunogenic protein aggregates.

38. The pharmaceutical composition according to claim 35, wherein the protein is solubilized in a pharmaceutically acceptable carrier having a pH value selected in a group consisting of (i) a pH value of 0.2 pH units or more higher than the isoelectric point of the said protein active ingredient, and (ii) a pH value of 0.2 pH units or less lower than the isoelectric point of the said protein active ingredient.

39. The pharmaceutical composition according to claim 35, wherein the protein is solubilized in a pharmaceutically acceptable carrier having a pH value of 0.2 pH units or less lower than the isoelectric point of the said protein active ingredient.

40. The pharmaceutical composition according to claim 35, wherein the pharmaceutically acceptable carrier comprises at least two amino acids having opposite charges, advantageously one or more basic amino acid and one or more acidic amino acid, and preferably arginine and glutamate.

41. The pharmaceutical composition according to claim 35, wherein the said one or more proteins are recombinant proteins.

42. The pharmaceutical composition according to claim 35, wherein the said one or more proteins are selected in a group consisting of:
a cytokine, which includes: an interleukin such as IL-7, IL-2, IL-21, IL-15, IL-12 and an interferon, such as interferon α, β, δ, γ, λ and their close analogs; or
a fusion protein comprising a cytokine or the soluble receptor of a cytokine (interleukin or interferon) and the Fc fragment of an immunoglobulin; or
an immuno-activating monoclonal antibody selected in a group consisting of anti-PD1, anti-PDL1, anti-CTLA-4, anti-Lag3, anti-Tim3 and anti-TGFβ or
a human growth hormone; or
an anti-hemophilic factor selected in a group consisting of factor VII and VIII.

43. A method for the chronic treatment of diseases comprising a step of administering a pharmaceutical composition according to claim 35.
